(19) United States
(12) Patent Application Publication
(10) Pub. No.: US 2009/0099217 A1
(43) Pub. Date: Apr. 16, 2009

(54) 2-Aminopyrimidin-4-Ones And Their Use For Treating Or Preventing Alpha Beta-Related Pathologies

(75) Inventors: Stefan Berg, Sodertalje (SE); Johan Hedstrom, Sodertalje (SE); Sven Hellberg, Sodertalje (SE); Johan Malmberg, Sodertalje (SE)

Correspondence Address: Pepper Hamilton LLP, 400 Berwyn Park, 899 Cassatt Road, Berwyn, PA, 19312-1183, US

(73) Assignees: ASTEX THERAPEUTICS LTD., Cambridge (GB); ASTRAECA AB, dertalje (SE)

(21) Appl. No.: 12/295,959

(22) PCT Filed: Apr. 4, 2007
(86) PCT No.: PCT/SE2007/000322

§ 371 Date: Oct. 3, 2008

Related U.S. Application Data
(60) Provisional application No. 60/789,540, filed on Apr. 5, 2006.

(51) Int. Cl. A61K 31/505; C07D 239/12; A61P 25/00

(52) U.S. Cl. 514/272; 544/321

(57) ABSTRACT

This invention relates to novel compounds having the structural formula (I) below: [Chemical formula should be inserted here. Please see paper copy] and to their pharmaceutically acceptable salt, compositions and methods of use. These novel compounds provide a treatment or prophylaxis of cognitive impairment, Alzheimer Disease, neurodegeneration and dementia.

The present invention relates to novel compounds, their pharmaceutical compositions. In addition, the present invention relates to therapeutic methods for the treatment and/or prevention of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

### BACKGROUND OF THE INVENTION

Several groups have identified and isolated aspartate proteinases that have β-secretase activity (Hussain et al., 1999; Lin et. al, 2000; Yan et. al, 1999; Sinha et. al., 1999 and Vassar et. al., 1999). β-secretase is also known in the literature as Asp2 (Yan et. al, 1999), Beta site APP Cleaving Enzyme (BACE) (Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase. Mention is also made of the patent literature: WO96/40885, EP871720, U.S. Pat. Nos. 5,942,400 and 5,744,346, EP855444, U.S. Pat. No. 6,319,689, WO99/64587, WO99/31236, EP1037977, WO00/17369, WO01/23533, WO0047618, WO00/58479, WO00/69262, WO01/00663, WO01/00665, U.S. Pat. No. 6,313,268.

BACE was found to be a pepsin-like aspartic proteinase, the mature enzyme consisting of the N-terminal catalytic domain, a transmembrane domain, and a small cytoplasmic domain. BACE has an optimum activity at pH 4.0-5.0 (Vassar et al, 1999)) and is inhibited weakly by standard pepsin inhibitors such as pepstatin. It has been shown that the catalytic domain minus the transmembrane and cytoplasmic domain has activity against substrate peptides (Lin et al, 2000). BACE is a membrane bound type 1 protein that is synthesized as a partially active proenzyme, and is abundantly expressed in brain tissue. It is thought to represent the major β-secretase activity, and is considered to be the rate-limiting step in the production of amyloid-β-protein (Aβ). It is thus of special interest in the pathology of Alzheimer's disease, and in the development of drugs as a treatment for Alzheimer's disease.

Aβ or amyloid-β-protein is the major constituent of the brain plaques which are characteristic of Alzheimer's disease (De Strooper et al, 1999). Aβ is a 39-42 residue peptide formed by the specific cleavage of a class I transmembrane protein called APP, or amyloid precursor protein. Aβ-secretase activity cleaves this protein between residues Met671 and Asp672 (numbering of 770aa isoform of APP) to form the N-terminus of Aβ. A second cleavage of the peptide is associated with γ-secretase to form the C-terminus of the Aβ peptide.

Alzheimer's disease (AD) is estimated to afflict more than 20 million people worldwide and is believed to be the most common form of dementia. Alzheimer's disease is a progressive dementia in which massive deposits of aggregated protein breakdown products—amyloid plaques and neurofibrillary tangles accumulate in the brain. The amyloid plaques are thought to be responsible for the mental decline seen in Alzheimer's patients.

The likelihood of developing Alzheimer's disease increases with age, and as the aging population of the developed world increases, this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much greater chance of developing AD, and also of developing it at an early age (see also U.S. Pat. No. 6,245,964 and U.S. Pat. No. 5,877,399 pertaining to transgenic rodents comprising APP-Swedish). Consequently, there is also a strong need for developing a compound that can be used in a prophylactic fashion for these individuals.

The gene encoding APP is found on chromosome 21, which is also the chromosome found as an extra copy in Down's syndrome. Down's syndrome patients tend to acquire Alzheimer's disease at an early age, with almost all those over 40 years of age showing Alzheimer's-type pathology (Oyama et al., 1994). This is thought to be due to the extra copy of the APP gene found in these patients, which leads to overexpression of APP and therefore to increased levels of APPβ causing the high prevalence of Alzheimer's disease seen in this population. Thus, inhibitors of BACE could be useful in reducing Alzheimer's-type pathology in Down's syndrome patients.

Drugs that reduce or block BACE activity should therefore reduce Aβ levels and levels of fragments of Aβ in the brain, or elsewhere where Aβ or fragments thereof deposit, and thus slow the formation of amyloid plaques and the progression of Aβ or other maladies involving deposition of Aβ or fragments thereof (Yankner, 1996; De Strooper and Konig, 1999). BACE is therefore an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

It would therefore be useful to inhibit the deposition of Aβ and portions thereof by inhibiting BACE through inhibitors such as the compounds provided herein.

The therapeutic potential of inhibiting the deposition of Aβ has motivated many groups to isolate and characterize secretase enzymes and to identify their potential inhibitors (see, e.g., WO01/23533 A2, EP0855444, WO00/17369, WO00/58479, WO00/47618, WO00/77030, WO01/00665, WO01/00663, WO01/29563, WO02/25276, U.S. Pat. No. 5,942,400, U.S. Pat. No. 6,245,884, U.S. Pat. No. 6,221,667, U.S. Pat. No. 6,211,235, WO02/02505, WO02/02506, WO02/02512, WO02/02518, WO02/02520, WO02/14264, WO05/058311, WO05/097767, WO06/041404, WO06/041404, WO06/0065204, US2006287294, WO06/138265, WO06/138217, WO06/138230, WO06/138264, WO06/138266, WO06/099379, US20070004786, US20070004730, WO07/011,833, WO07/011,810).

The compounds of the present invention show improved properties compared to the potential inhibitors known in the art, e.g. improved hERG selectivity.

### DISCLOSURE OF THE INVENTION

Provided herein are novel compounds of structural formula I:

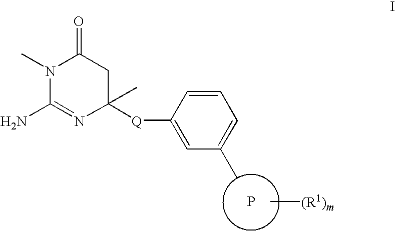

wherein

P is a pyridine ring;

Q is a bond or CH₂CH₂;

R¹ is independently selected from cyano, halogen, C₁₋₆alkyl and methoxy;

m is 1 or 2;

as a free base or a pharmaceutically acceptable salt, solvate or solvate of a salt thereof.

The present invention further provides pharmaceutical compositions comprising as active ingredient a therapeutically effective amount of a compound of formula I in association with pharmaceutically acceptable excipients, carriers or diluents.

The present invention further provides methods of modulating activity of BACE comprising contacting the BACE enzyme with a compound of formula I.

The present invention further provides methods of treating or preventing an Aβ-related pathology in a patient, comprising administering to the patient a therapeutically effective amount of a compound of formula I.

The present invention further provides a compound described herein for use as a medicament.

In one aspect of the present invention, there is provided a compound according to formula I:

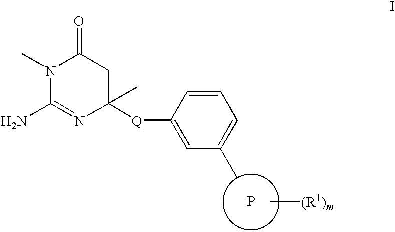

wherein

P is a pyridine ring;

Q is a bond or CH₂CH₂;

R¹ is independently selected from cyano, halogen, C₁₋₆alkyl and methoxy;

m is 1 or 2;

with the proviso that the following compounds are excluded;
2-Amino-6-[3-(5-bromopyridin-3-yl)phenyl]-3,6-dimethyl-5,6-dihydropyrimidin-4(3H)-one;
2-Amino-6-[3-(2-fluoropyridin-3-yl)phenyl]-3,6-dimethyl-5,6-dihydropyrimidin-4(3H)-one;
2-Amino-6-[3-(2-chloro-3-fluoropyridin-4-yl)phenyl]-3,6-dimethyl-5,6-dihydropyrimidin-4(3H)-one;
   as a free base or a pharmaceutically acceptable salt, solvate or solvate of a salt thereof.

In another aspect of the present invention, there is provided a compound according to formula I, wherein C₁₋₆alkyl represents methyl.

In another aspect of the present invention, there is provided a compound according to formula I, wherein Q represents a direct bond.

In another aspect of the present invention, there is provided a compound according to formula I, wherein m is 1 and R¹ is cyano, methoxy or halogen.

In one embodiment of this aspect R¹ is halogen, said halogen being a chloro attached in the 2-position in the pyridine ring.

In another aspect of the present invention, there is provided a compound according to formula I, wherein m is 2 and R¹ represents one halogen and one methyl.

In one embodiment of this aspect said halogen is a fluoro attached in the 6-position in the pyridine ring.

In another aspect of the present invention, there is provided a compound according to formula I, wherein m is 2 and R¹ represents two halogen atoms.

In one embodiment of this aspect said two halogen atoms represents either of the following combinations of halogens: chloro attached in the 2- and 3-position in the pyridine ring; fluoro attached in the 2-position and bromo attached in the 5-position in the pyridine ring; chloro attached in the 2-position and fluoro attached in the 5-position in the pyridine ring; fluoro attached in the 2-position and chloro attached in the 5-position in the pyridine ring.

In another aspect of the present invention, there is provided a compound according to formula I, wherein Q represents CH₂—CH₂.

In one embodiment of this aspect m is 1 and R¹ is selected from halogen or methoxy, for example chloro attached in the 2-position in the pyridine ring.

In another embodiment of this aspect m is 2 and R¹ represents two halogen atoms, for example fluoro attached in the 2-position and chloro attached in the 5-position in the pyridine ring.

In another aspect of the present invention, there is provided a compounds according to formula I, said compounds being:
5-[3-(2-Amino-1,4-dimethyl-6-oxo-1,4,5,6-tetrahydropyrimidin-4-yl)phenyl]nicotinonitrile hydrochloride;
2-Amino-6-[3-(2,3-dichloropyridin-4-yl)phenyl]-3,6-dimethyl-5,6-dihydropyrimidin-4(3H)-one acetate;
2-Amino-6-[3-(5-bromo-2-fluoropyridin-3-yl)phenyl]-3,6-dimethyl-5,6-dihydropyrimidin-4(3H)-one acetate;
2-Amino-6-[3-(2-chloro-5-fluoropyridin-4-yl)phenyl]-3,6-dimethyl-5,6-dihydropyrimidin-4(3H)-one trifluoroacetic acid salt;
2-Amino-6-[3-(5-chloro-2-fluoropyridin-3-yl)phenyl]-3,6-dimethyl-5,6-dihydropyrimidin-4(3H)-one hydrochloride;
2-Amino-6-[3-(6-fluoro-5-methylpyridin-3-yl)phenyl]-3,6-dimethyl-5,6-dihydropyrimidin-4(3H)-one hydrochloride;
2-Amino-6-[3-(2-chloropyridin-4-yl)phenyl]-3,6-dimethyl-5,6-dihydropyrimidin-4(3H)-one hydrochloride;
2-Amino-6-{2-[3-(2-chloropyridin-4-yl)phenyl]ethyl}-3,6-dimethyl-5,6-dihydropyrimidin-4(3H)-one hydrochloride;
2-Amino-6-[3-(5-methoxypyridin-3-yl)phenyl]-3,6-dimethyl-5,6-dihydropyrimidin-4(3H)-one hydrochloride;
2-Amino-6-{2-[3-(6-methoxypyridin-2-yl)phenyl]ethyl}-3,6-dimethyl-5,6-dihydropyrimidin-4(3H)-one hydrochloride; and
2-Amino-6-{2-[3-(5-chloro-2-fluoropyridin-3-yl)phenyl]ethyl}-3,6-dimethyl-5,6-dihydropyrimidin-4(3H)-one hydrochloride;
   as a free base or a pharmaceutically acceptable salt, solvate or solvate of a salt thereof.

Some compounds of formula I may have stereogenic centres and/or geometric isomeric centres (E- and Z-isomers), and it is to be understood that the invention encompasses all such optical isomers, enantiomers, diastereoisomers, atropisomers and geometric isomers.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

It is to be understood that the present invention relates to any and all tautomeric forms of the compounds of formula I.

Compounds of the invention can be used as medicaments. In some embodiments, the present invention provides compounds of formula I, or pharmaceutically acceptable salts, tautomers or in vivo-hydrolysable precursors thereof, for use as medicaments. In some embodiments, the present invention provides compounds described here in for use as medicaments for treating or preventing an Aβ-related pathology. In some further embodiments, the Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In some embodiments, the present invention provides use of compounds of formula I or pharmaceutically acceptable salts, tautomers or in vivo-hydrolysable precursors thereof, in the manufacture of a medicament for the treatment or prophylaxis of Aβ-related pathologies. In some further embodiments, the Aβ-related pathologies include such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method of inhibiting activity of BACE comprising contacting the BACE with a compound of the present invention. BACE is thought to represent the major β-secretase activity, and is considered to be the rate-limiting step in the production of amyloid-β-protein (Aβ). Thus, inhibiting BACE through inhibitors such as the compounds provided herein would be useful to inhibit the deposition of Aβ and portions thereof. Because the deposition of Aβ and portions thereof is linked to diseases such Alzheimer Disease, BACE is an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

In some embodiments, the present invention provides a method for the treatment of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, comprising administering to a mammal (including human) a therapeutically effective amount of a compound of formula I, or a pharmaceutically acceptable salt, tautomer or in vivo-hydrolysable precursor thereof.

In some embodiments, the present invention provides a method for the prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration comprising administering to a mammal (including human) a therapeutically effective amount of a compound of formula Ia or a pharmaceutically acceptable salt, tautomer or in vivo-hydrolysable precursors.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula I or a pharmaceutically acceptable salt, tautomer or in vivo-hydrolysable precursors and a cognitive and/or memory enhancing agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration by administering to a mammal (including human) a compound of formula I or a pharmaceutically acceptable salt, tautomer or in vivo-hydrolysable precursors thereof wherein constituent members are provided herein, and a choline esterase inhibitor or anti-inflammatory agent.

In some embodiments, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, or any other disease, disorder, or condition described herein, by administering to a mammal (including human) a compound of the present invention and an atypical antipsychotic agent. Atypical antipsychotic agents includes, but not limited to, Olanzapine (marketed as Zyprexa), Aripiprazole (marketed as Abilify), Risperidone (marketed as Risperdal), Quetiapine (marketed as Seroquel), Clozapine (marketed as Clozaril), Ziprasidone (marketed as Geodon) and Olanzapine/Fluoxetine (marketed as Symbyax).

In some embodiments, the mammal or human being treated with a compound of the invention has been diagnosed with a particular disease or disorder, such as those described herein. In these cases, the mammal or human being treated is in need of such treatment. Diagnosis, however, need not be previously performed.

The present invention also includes pharmaceutical compositions which contain, as the active ingredient, one or more of the compounds of the invention herein together with at least one pharmaceutically acceptable carrier, diluent or excipient.

The definitions set forth in this application are intended to clarify terms used throughout this application. The term “herein” means the entire application.

A variety of compounds in the present invention may exist in particular geometric or stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms, by synthesis from optically active starting materials, or synthesis using optically active reagents. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross, a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents, positions of substituents and/or variables are permissible only if such combinations result in stable compounds.

As used herein, “alky” used alone or as a suffix or prefix, is intended to include both branched and straight-chain saturated aliphatic hydrocarbon groups having from 1 to 12 carbon atoms or if a specified number of carbon atoms is provided then that specific number would be intended. For example “C₁₋₆ alkyl” denotes alkyl having 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl.

As used herein, “halo” or “halogen” refers to fluoro, chloro, bromo, and iodo.

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric acid.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like diethyl ether, ethyl acetate, ethanol, isopropanol, or acetonitrile are used.

As used herein, “in vivo hydrolysable precursors” means an in vivo hydroysable (or cleavable) ester of a compound of Formula (I) that contains a carboxy or a hydroxy group. For example amino acid esters, C₁₋₆ alkoxymethyl esters like methoxymethyl; C₁₋₆alkanoyloxymethyl esters like pivaloyloxymethyl; C₃₋₈cycloalkoxycarbonyloxy C₁₋₆alkyl esters like 1-cyclohexylcarbonyloxyethyl, acetoxymethoxy, or phosphoramidic cyclic esters.

As used herein, “tautomer” means other structural isomers that exist in equilibrium resulting from the migration of a hydrogen atom. For example, keto-enol tautomerism where the resulting compound has the properties of both a ketone and an unsaturated alcohol.

As used herein “stable compound” and “stable structure” are meant to indicate a compound that is sufficiently robust to survive isolation to a useful degree of purity from a reaction mixture, and formulation into an efficacious therapeutic agent.

Compounds of the invention further include hydrates and solvates.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to 2H (also written as D for deuterium), ³H (also written as T for tritium), ¹¹C, ¹³c, ¹⁴c, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radio nuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from the group consisting of ³H, ¹⁴c, ¹²⁵I, ³⁵S and ⁸²Br.

The anti-dementia treatment defined herein may be applied as a sole therapy or may involve, in addition to the compound of the invention, conventional chemotherapy. Such chemotherapy may include one or more of the following categories of agents: acetyl cholinesterase inhibitors, anti-inflammatory agents, cognitive and/or memory enhancing agents or atypical antipsychotic agents.

Such conjoint treatment may be achieved by way of the simultaneous, sequential or separate dosing of the individual components of the treatment. Such combination products employ the compounds of this invention.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of dementia is an amount sufficient to symptomatically relieve in a warm-blooded animal, particularly a human the symptoms of dementia, to slow the progression of dementia, or to reduce in patients with symptoms of dementia the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

In some embodiments, the present invention provides a compound of formula I or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The compounds of the invention may be derivatised in various ways. As used herein “derivatives” of the compounds includes salts (e.g. pharmaceutically acceptable salts), any complexes (e.g. inclusion complexes or clathrates with compounds such as cyclodextrins, or coordination complexes with metal ions such as Mn²⁺ and Zn²⁺), free acids or bases, polymorphic forms of the compounds, solvates (e.g. hydrates), prodrugs or lipids, coupling partners and protecting groups. By “prodrugs” is meant for example any compound that is converted in vivo into a biologically active compound.

Salts of the compounds of the invention are preferably physiologically well tolerated and non toxic. Many examples of salts are known to those skilled in the art. All such salts are within the scope of this invention, and references to compounds include the salt forms of the compounds.

Where the compounds contain an amine function, these may form quaternary ammonium salts, for example by reaction with an alkylating agent according to methods well known to the skilled person. Such quaternary ammonium compounds are within the scope of the invention.

Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Where the compounds contain chiral centres, all individual optical forms such as enantiomers, epimers and diastereoisomers, as well as racemic mixtures of the compounds are within the scope of the invention.

Compounds may exist in a number of different geometric isomeric, and tautomeric forms and references to compounds include all such forms. For the avoidance of doubt, where a compound can exist in one of several geometric isomeric or tautomeric forms and only one is specifically described or shown, all others are nevertheless embraced by the scope of this invention.

The quantity of the compound to be administered will vary for the patient being treated and will vary from about 100 ng/kg of body weight to 100 mg/kg of body weight per day and preferably will be from 10 pg/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

Compounds of the present invention have been shown to inhibit beta secretase (including BACE) activity in vitro. Inhibitors of beta secretase have been shown to be useful in blocking formation or aggregation of Aβ peptide and therefore have beneficial effects in treatment of Alzheimer's Disease and other neurodegenerative diseases associated with elevated levels and/or deposition of Aβ peptide. Therefore, it is believed that the compounds of the present invention may be used for the treatment of Alzheimer disease and disease associated with dementia. Hence, compounds of the present invention and their salts are expected to be active against age-related diseases such as Alzheimer, as well as other Aβ related pathologies such as Downs syndrome and β-amyloid angiopathy. It is expected that the compounds of the present invention would most likely be used as single agents but could also be used in combination with a broad range of cognition deficit enhancement agents.

### General Methods

Starting materials used were available from commercial sources, or prepared according to literature procedures.

Microwave heating was performed in a Creator, Initiator or Smith Synthesizer Single-mode microwave cavity producing continuous irradiation at 2450 MHz.

¹H NMR spectra were recorded in the indicated deuterated solvent at either 300 MHz, 400 MHz, 500 MHz, or 600 MHz. The 400 MHz spectra were obtained unless stated otherwise, using a Bruker av400 NMR spectrometer equipped with a 3 mm flow injection SEI ¹H/D-¹³C probe head with Z-gradients, using a BEST 215 liquid handler for sample injection, or using a Bruker DPX400 NMR spectrometer equipped with a 4-nucleus probehead with Z-gradients. 600 MHz ¹H NMR were recorded using a Bruker DRX600 NMR spectrometer equipped with a 5 mm TXI probehead with Z-gradients. 500 MHz ¹H NMR were recorded using a Varian INOVA, (magnet: Oxford AS500) 500 NMR spectrometer. Chemical shifts are given in ppm down- and upfield from TMS. Resonance multiplicities are denoted s, d, t, q, m and br for singlet, doublet, triplet, quartet, multiplet, and broad respectively.

LC-MS analyses were recorded on a Waters LCMS equipped with a Waters X-Terra MS, C8-column, (3.5 μm, 100 mm×3.0 mm i.d.). The mobile phase system consisted of A: 10 mM ammonium acetate in water/acetonitrile (95:5) and B: acetonitrile. A linear gradient was applied running from 0% to 100% B in 4-5 minutes with a flow rate of 1.0 mL/min. The mass spectrometer was equipped with an electrospray ion source (ESI) operated in a positive or negative ion mode. The capillary voltage was 3 kV and the mass spectrometer was typically scanned between m/z 100-700. Alternative, LC-MS HPLC conditions were as follows: Column: Agilent Zorbax SB-C8 2 mm ID×50 mm Flow: 1.4 mL/minGradient: 95% A to 90% B over 3 min. hold 1 minute ramp down to 95% A over 1 minute and hold 1 minute. Where A=2% acetonitrile in water with 0.1% formic acid and B=2% water in acetonitrile with 0.1% formic acid. UV-DAD 210-400 nm Mass spectra (MS) were run using an automated system with atmospheric pressure chemical (APCI or CI) or electrospray (+ESI) ionization. Generally, only spectra where parent masses are observed are reported. The lowest mass major ion is reported for molecules where isotope splitting results in multiple mass spectral peaks (for example when chlorine is present).

Thin layer chromatography (TLC) was performed on Merch TLC-plates (Silica gel 60 F₂₅₄) and spots were UV visualized. Flash chromatography was performed using Merck Silica gel 60 (0.040-0.063 mm), or employing a Combi Flash® Companion™ system using RediSep™ normal-phase flash columns.

Compounds have been named using ACD/Name, version 8.08 or 9.0, software from Advanced Chemistry Development, Inc. (ACD/Labs), Toronto ON, Canada, www.acdlabs.com, 2004.

### EXAMPLES

Below follows a number of non-limiting examples of compounds of the invention.

### Example 1

### tert-Butyl (2E)-3-(3-bromophenyl)but-2-enoate

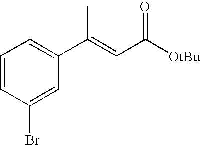

To a −78° C. stirred solution of tert-butyldimethylphosphonoacetate (21.9 mL, 0.111 mol) in tetrahydrofuran (150 mL) was added n-butyl lithium in hexane (1.6 M, 72.0 mL, 0.116 mol) and the reaction was stirred at −78° C. for 10 min. To this mixture was added 3′-bromoacetophenone (13.4 mL, 0.100 mol) and the reaction was allowed to warm to room temperature and stirred for 18 h. The tetrahydrofuran was removed under reduced pressure to yield a solid. Hexane (300 μL) was added and the solids triturated for one hour. The mixture was filtered through Celite and the filtrate concentrated under reduced pressure to give 28.9 g the title compound. This was carried directly into the next reaction: ¹H NMR (300 MHz, DMSO-d₆): δ 1.47 (s, 9H); 2.44 (s, 3H); 6.05 (s, 1H); 7.36 (t, J=7.8 Hz, 1H); 7.53 (m, 2H); 7.71 (s, 1H).

### Example 2

### (2E)-3-(3-Bromophenyl)but-2-enoic acid

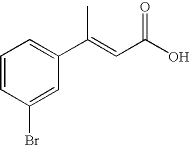

A solution of crude tert-butyl (2E)-3-(3-bromophenyl)but-2-enoate (28.9 g) in trifluororacetic acid: dichloromethane (1:1, 300 mL) was stirred at room temperature for 15 min and the solvents were removed under reduced pressure. The crude solid was triturated in hexane (400 mL), filtered, and dried under vacuum to give 8.87 g (38% yield) of the title compound: ¹H NMR (300 MHz, DMSO-d₆): δ 2.46 (s, 3H); 6.11 (s, 1H); 7.37 (t, J=7.8 Hz, 1H); 7.53 (m, 2H); 7.72 (t, J=1.5 Hz, 1H).

### Example 3

### (2E)-3-(3-Bromophenyl)but-2-enoyl chloride

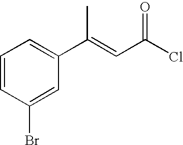

To a suspension of (2E)-3-(3-bromophenyl)but-2-enoic acid (1.0 g, 4.15 mmol) in dichloromethane (10 mL) was added oxalyl chloride (434 μL, 4.98 mmol) followed by N,N-dimethylformamide (15 μL, 0.207 mmol) and the reaction was stirred at room temperature. After 2 h the solvent was removed under reduced pressure to give the title compound: ¹H NMR (300 MHz, DMSO-d₆): δ 2.51 (s, 3H); 6.44 (s, 1H); 7.29 (t, J=7.8 Hz, 1H); 7.43 (d, J=7.8 Hz, 1H); 7.57 (d, J=8.7 Hz, 1H); 7.63 (t, J=1.8 Hz, 1H).

### Example 4

### (2E)-3-(3-Bromophenyl)-N-cyano-N-methylbut-2-enamide

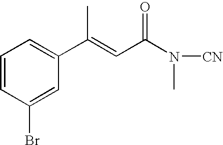

To a −60° C. stirred solution of cyanogen bromide (4.24 g, 40.0 mmol) in tetrahydrofuran (100 mL) was added sodium carbonate (6.36 g, 60.0 mmol) followed by drop wise addition of a solution of methyl amine in tetrahydrofuran (2.0 M, 20.0 mL 40.0 mmol). The bath temperature was kept below −20° C. for 2 h. The reaction was filtered cold under a blanket of nitrogen through Celite and a solution (2E)-3-(3-bromophenyl)but-2-enoyl chloride (5.19 g, 20.00 mmol) in tetrahydrofuran (100 mL) was added to the filtrate. To this mixture was added N,N-diisopropylethylamine (4.2 mL, 24.0 mmol) and the reaction was stirred at room temperature for 2 h. The solvent was removed under reduced pressure and the resulting oil put under high vacuum over night. The crude compound was purified on a silica gel column eluting with dichloromethane to give 4.29 g (75% yield) of the title compound: ¹H NMR (300 MHz, DMSO-d₆): δ 2.44 (s, 3H); 3.22 (s, 3H); 6.65 (s, 1H); 7.42 (t, J=7.8 Hz, 1H); 7.58 (d, J=8.4 Hz, 1H); 7.65 (d, J=7.8 Hz, 1H); 7.76 (t, J=1.8 Hz, 1H).

### Example 5

### 6-(3-Bromophenyl)-2-imino-1-(4-methoxybenzyl)-3,6-dimethyltetrahydropyrimidin-4(1H)-one

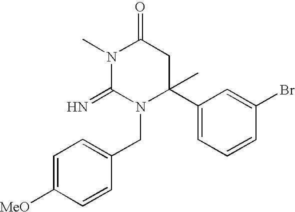

To a solution of (2E)-3-(3-bromophenyl)-N-cyano-N-methylbut-2-enamide (12.77 g, 45.75 mmol) in N,N-dimethylformamide (50 mL) was added 4-methoxybenzyl amine (14.9 mL, 114.38 mmol). After 4 h, the solvent was removed under reduced pressure and the resulting viscous oil put under high vacuum over night. The crude compound was purified using sequential column chromatography on silica gel. The first purification was eluting with dichloromethane and methanol (2.5:97.5)/dichloromethane, methanol/dichloromethane (5:95) to give 18.96 g of the crude product. The second purification was eluting with diethyl ether, ethyl acetate, methanol/ethyl acetate (5:95), methanol/ethyl acetate (10:90) to give 15.48 gram (81% yield) of the title compound: ¹H NMR (300 MHz, DMSO-d₆/TFA-d): δ 1.65 (s, 3H); 3.20 (s, 3H); 3.30 (d, J=16.5 Hz, 1H); 3.58 (d, J=16.8 Hz, 1H); 3.78 (s, 3H); 4.97 (dd, J=4.8 Hz, 2H); 6.96 (d, J=8.7 Hz, 2H); 7.34 (m, 4H); 7.57 (m, 2H); MS (APCI+) m/z 416.08 [M+1]⁺.

### Example 6

### 2-Amino-6-(3-bromophenyl)-3,6-dimethyl-5,6-dihydropyrimidin-4(3H)-one

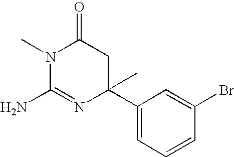

To a solution of 6-(3-bromophenyl)-2-imino-1-(4-methoxybenzyl)-3,6-dimethyltetrahydropyrimidin-4(1H)-one (15.48 g, 37.18 mmol) in acetonitril (150 mL) was added water (50 mL) followed by ammonium cerium nitrate (61.15 g, 111.55 mmol) and the reaction was stirred for 18 h. Celite (32 g) was added followed by sodium bicarbonate (31.23 g, 371.8 mmol) and the reaction stirred for 2 h. Additional Celite (15 g) was added after 1 h. The reaction was filtered through Celite and the filtrate was concentrated under reduced pressure. The resulting orange solid was put under high vacuum. A crude purification was done on a silica gel column using methanol/dichloromethan/acetic acid (15:85:0.1) as the eluent. The resulting orange solid was triturated with methanol to give the first batch of the title compound. The solvents were removed from the filtrate under reduced pressure and the resulting orange solid was triturated with ethanol to give a second batch of the title compound. The batches were combined to give 8.75 g (79% yield) of the title compound: ¹H NMR (300 MHz, DMSO-d₆/TFA-d): δ 1.64 (s, 3H); 3.14 (s, 3H); 3.19 (d, J=16.5 Hz, 1H); 3.49 (d, J=16.2 Hz, 1H); 7.39 (m, 2H); 7.55 (m, 1H); 7.67 (s, 1H); MS (APCI+) m/z 296.0 [M+1]⁺.

### Example 7

### 5-[3-(2-Amino-1,4-dimethyl-6-oxo-1,4,5,6-tetrahydropyrimidin-4-yl)phenyl]nicotinonitrile hydrochloride

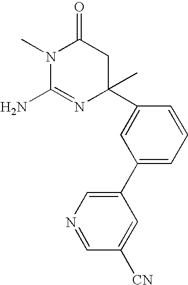

Triisopropyl borate (0.33 mL, 1.44 mmol) was added to a stirred solution of 5-bromonicotinonitrile (0.088 g, 0.48 mmol) in anhydrous tetrahydrofuran (5 mL). The resulting solution was cooled to −70° C. and n-butyllithium (0.59 mL, 2.5 M in hexane, 1.49 mmol) was added dropwise. The mixture was stirred at −70° C. for 1 h and was then allowed to warm to room temperature. Aqueous hydrochloric acid (1.2 mL, 2 M, 2.4 mmol) was added and the mixture was stirred for 5 min, followed by addition of aqueous sodium carbonate (2.4 μL, 2 M, 4.8 mmol). After stirring for another 10 min, 2-amino-6-(3-bromophenyl)-3,6-dimethyl-5,6-dihydropyrimidin-4(3H)-one (0.100 g, 0.34 mmol) and [1,1′-bis(diphenylphosphino)ferrocene]palladium(II) chloride dichloromethane adduct (0.047 g, 0.057 mmol) were added and the resulting mixture was heated at 65° C. overnight. The solvent was evaporated and the residue was purified on a silica gel column using acetonitrile/triethylamine, (95:5), as the eluent. Additional purification was made by preparative HPLC, using a XTerra column (19×300 mm) (0.1 M aqueous ammonium acetate in acetonitrile (10 to 50% acetonitrile)), as the eluent, to give 0.022 g of the base. The base was dissolved in chloroform and methanol and hydrochloric acid (4 M in diethyl ether) was added followed by addition of diethyl ether until precipitation. The solid was collected by filtration and dried in vacuo at 70° C. to give 10 mg (8.3% yield) of the title compound: ¹H NMR (DMSO-d₆, 400 MHz) δ 10.43 (s, 1H), 9.24 (d, J=2.3 Hz, 1H), 9.05 (d, J=1.8 Hz, 1H), 8.71 (t, J=2.0 Hz, 1H), 8.72-8.62 (m, 2H), 7.92 (s, 1H), 7.82 (d, J=7.8 Hz, 1H), 7.59 (t, J=7.8 Hz, 1H), 7.50 (d, J=8.0 Hz, 1H), 3.58 (d, J=16.3 Hz, 1H), 3.22 (d, J=16.6 Hz, 1H), 3.09 (s, 3H), 1.67 (s, 3H).

### Example 8

### 2-Amino-6-[3-(2,3-dichloropyridin-4-yl)phenyl]-3,6-dimethyl-5,6-dihydropyrimidin-4(3H)-one acetate

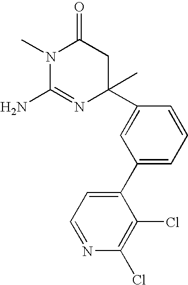

2-Amino-6-(3-bromophenyl)-3,6-dimethyl-5,6-dihydropyrimidin-4(3H)-one (0.100 g, 0.34 mmol), (2,3-dichloropyridin-4-yl)boronic acid (0.090 g, 0.47 mmol), [1,1′-bis(diphenylphosphino)ferrocene]palladium(II) chloride dichloromethane adduct (0.027 g, 0.034 mmol) and aqueous sodium carbonate (0.59 mL, 2 M, 1.18 mmol) were dissolved in a mixture of tetrahydrofuran and water (9:1, 3 mL) and heated at 140° C. for 10 min by microwave irradiation. The solvent was evaporated, the residue was partitioned between water and dichloromethane (3×10 mL). The combined extracts were dried over sodium sulfate, and the solvents were removed in vacuo to afford 0.1 g. The residue was purified by prep. HPLC, using a XTerra column (19×300 mm) with 0.1 M ammonium acetate buffer/acetonitrile, (25-65% acetonitrile), as a eluent. Fractions containing product were collected and evaporated in vacuo and dried at 25° C. in a vacuum-cabinet over night to give 21 mg (15% yield) of the title compound: ¹H NMR (DMSO-d₆, 400 MHz) δ 8.44 (d, J=4.8 Hz, 1H), 7.62-7.59 (m, 1H), 7.57 (d, J=8.3 Hz, 1H), 7.50-7.44 (m, 2H), 7.36 (d, J=7.8 Hz, 1H), 3.00 (s, 3H), 2.92 (d, J=16.1 Hz, 1H), 2.78 (d, J=16.1 Hz, 1H), 1.89 (s, 3H), 1.41 (s, 3H).

### Example 9

### 2-Amino-6-[3-(5-bromo-2-fluoropyridin-3-yl)phenyl]-3,6-dimethyl-5,6-dihydropyrimidin-4(3H)-one acetate

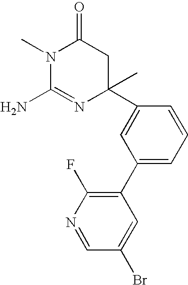

The title compound was synthesized as described for Example 8 starting from 2-amino-6-(3-bromophenyl)-3,6-dimethyl-5,6-dihydropyrimidin-4(3H)-one and (5-bromo-2-fluoropyridin-3-yl)boronic acid to give 5 mg (3% yield) of the title compound: ¹H NMR (DMSO-d₆, 400 MHz) δ 8.40-8.38 (m, 1H), 8.34 (dd, J=8.8, 2.5 Hz, 1H), 7.73-7.69 (m, 1H), 7.57-7.53 (m, 1H), 7.51-7.42 (m, 2H), 3.00 (s, 3H), 2.91 (d, J=15.8 Hz, 1H), 2.78 (d, J=16.1 Hz, 1H), 1.89 (s, 2H), 1.41 (s, 3H).

### Example 10

### 2-Amino-6-[3-(2-chloro-5-fluoropyridin-4-yl)phenyl]-3,6-dimethyl-5,6-dihydropyrimidin-4(3H)-one trifluoroacetate

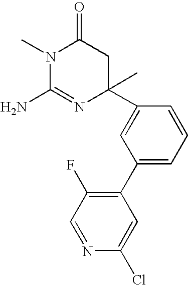

The title compound was synthesized as described for Example 8 starting from 2-amino-6-(3-bromophenyl)-3,6-dimethyl-5,6-dihydropyrimidin-4(3H)-one and (2-chloro-5-fluoropyridin-4-yl)boronic acid. Purified by prep. HPLC, using a XTerra column (19×300 mm) with 0.1% trifluoroacetic acid buffer/methanol, (20-60% methanol), as a eluent, to give 16 mg (5% yield) of the title compound: ¹H NMR (DMSO-d₆, 400 MHz) δ 10.33 (br s, 1H), 8.66 (br s, 2H), 8.57 (d, J=2.0 Hz, 1H), 7.80 (d, J=5.8 Hz, 1H), 7.73-7.69 (m, 1H), 7.69-7.64 (m, 1H), 7.60 (t, J=7.6 Hz, 1H), 7.57-7.53 (m, 1H), 3.53 (d, J=16.6 Hz, 1H), 3.21 (d, J=16.3 Hz, 1H), 3.08 (s, 3H), 1.67 (s, 3H).

### Example 11

### 4-(3-Bromophenyl)butan-2-one

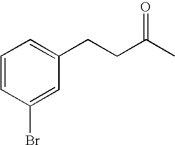

To 2,4-pentanedione (15.8 mL, 0.154 mol) in ethanol (150 mL) was added potassium carbonate (19.35 g, 0.140 mol) followed by 3-bromobenzyl bromide (35.00 g, 0.140 mol) and the mixture was heated to reflux. After refluxing for 18 h the reaction was cooled and the salts removed by filtration. The filtrate was evaporated and ethyl acetate and water (200:100 mL) was added. The ethyl acetate layer was washed twice with aqueous hydrochloric acid (1 M, 100 mL), once with saturated aqueous sodium bicarbonate (100 mL) and once with saturated aqueous sodium chloride (100 mL) and dried over sodium sulfate. The ethyl acetate was removed under reduced pressure and the resulting yellow oil was put under high vacuum. The oil was purified on silica gel in a filter funnel (0.5 kg) eluting with 1000 mL each of a 5% step gradient 0-100% hexane/dichloromethane. The purified product was removed of solvent under reduced pressure to give 18.5 g (58% yield) of the title compound: ¹H NMR (300 MHz, DMSO-d₆) δ 2.10 (s, 3H), 2.79 (s, 4H), 7.22-7.24 (m, 2H), 7.35-7.39 (m, 1H), 7.43 (s, 1H).

### Example 12

### tert-Butyl (2E)-5-(3-bromophenyl)-3-methylpent-2-enoate

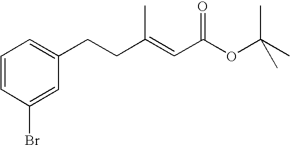

To a −78° C. stirred solution of tert-butyldimethylphosphonoacetate (19.5 mL, 89.6 mmol) in tetrahydrofuran (100 mL) was added n-butyllithium in hexane (1.6 M, 35.8 mL, 89.6 mmol) and the reaction was stirred at −78° C. for 25 min. To this mixture was added 4-(3-bromophenyl)butan-2-one (18.50 g, 81.46 mmol) and the reaction was allowed to warm to room temperature and stirred for 1.5 h. The tetrahydrofuran was removed under reduced pressure to yield a solid. To this was added hexane (300 mL) and the solids triturated for 1 h. The mixture was filtered through Celite and the filtrate concentrated under reduced pressure to give the title compound: ¹H NMR (300 MHz, DMSO-d₆) δ 1.42 (d, J=3.2 Hz, 9H), 1.86 (d, J=1.3 Hz, 1H), 2.12 (d, J=1.1 Hz, 2H), 2.37-2.43 (m, 2H), 2.73-2.78 (m, 2H), 5.58-5.60 (m, 1H), 7.24 (d, J=5.2 Hz, 2H), 7.35-7.40 (m, 1H), 7.45 (d, J=5.4 Hz, 1H).

### Example 13

### (2E)-5-(3-Bromophenyl)-3-methylpent-2-enoic acid

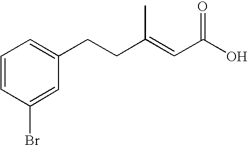

A solution of crude tert-butyl (2E)-5-(3-bromophenyl)-3-methylpent-2-enoate in trifluororacetic acid/dichloromethane (1:1, 100 mL) was stirred at room temperature for 15 min and the solvents removed under reduced pressure. The crude solid was triturated in hexane (100 mL), filtered, and dried under vacuum to give 18.48 g (84% yield) of the title compound: ¹H NMR (300 MHz, DMSO-d₆) δ 1.88 (d, J=1.3 Hz, 1H), 2.14 (d, J=1.1 Hz, 2H), 2.43 (t, J=8.1 Hz, 2H), 2.71-2.80 (m, 2H), 5.62 (d, J=1.1 Hz, 5H), 5.66 (s, 5H), 7.25 (d, J=4.5 Hz, 2H), 7.35-7.42 (m, 1H), 7.47 (s, 1H).

### Example 14

### (2E)-5-(3-Bromophenyl)-N-cyano-N,3-dimethylpent-2-enamide

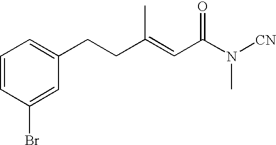

To a 0° C. stirred solution of (2E)-5-(3-bromophenyl)-3-methylpent-2-enoic acid (18.48 g, 68.66 mmol) in dichloromethane (100 mL) was added oxalyl chloride (7.2 mL, 82.10 mmol) followed by N,N-dimethylformamide (0.266 mL, 3.43 mmol) and the reaction was kept cold for 1 h, warmed to room temperature and stirred for another hour. The solvent was removed under reduced pressure to give 21.78 g of the corresponding acid chloride.

To a −78° C. stirred solution of cyanogen bromide (14.55 g, 0.137 mmol) in tetrahydrofuran (100 mL) was added sodium carbonate (21.84 g, 0.206 mmol) followed by dropwise addition of a solution of methyl amine in tetrahydrofuran (2.0 M, 68.7 mL, 0.137 mol). The mixture was stirred at −78° C. for 1.5 h. The reaction was filtered cold under a blanket of nitrogen through Celite and a solution of the above formed acid chloride (19.75 g, 0.0686 mmol) in tetrahydrofuran (100 mL) was added to the filtrate. To this solution was added N,N-diisopropylethylamine (14.4 mL, 0.082 mol) and the reaction was stirred at room temperature for 2 h. The solvent was removed under reduced pressure and the resulting oil put under high vacuum over night. The crude compound was purified on a silica gel column eluting with dichloromethane to give 16.64 g (79% yield) of the title compound: ¹H NMR (300 MHz, DMSO-d₆) δ 1.99 (d, J=1.3 Hz, 1H), 2.14 (d, J=1.1 Hz, 2H), 2.52 (dd, J=3.8, 2.0 Hz, 2H), 2.79 (t, J=7.7 Hz, 2H), 3.13 (s, 3H), 6.22 (d, J=6.6 Hz, 1H), 7.24-7.27 (m, 2H), 7.37-7.41 (m, 1H), 7.49 (s, 1H); MS (APCI+) m/z 307) [M+1]⁺.

### Example 15

### 6-[2-(3-Bromophenyl)ethyl]-2-imino-1-(4-methoxybenzyl)-3,6-dimethyltetrahydropyrimidin-4(1H)-one

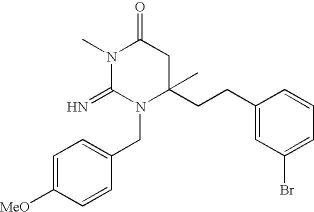

To a solution of (2E)-5-(3-bromophenyl)-N-cyano-N,3-dimethylpent-2-enamide (15.00 g, 48.83 mmol) in N,N-dimethylformamide (50 mL) was added 4-methoxybenzyl amine (12.8 mL, 97.66 mmol). After 4 h, the solvent was removed under reduced pressure and the resulting viscous oil put under high vacuum over night. The crude compound was purified by chromatography on silica gel by eluting with dichloromethane/methanol, (95:5), to give 20.45 g of the semi-pure title compound which was carried forward as is: ¹H NMR (300 MHz, DMSO-d₆/TFA-d) δ 1.34 (s, 3H), 1.93 (dd, J=65.3, 6.0 Hz, 2H), 2.55-2.64 (m, 2H), 3.05 (dd, J=24.0, 16.8 Hz, 2H), 3.27 (d, J=17.3 Hz, 3H), 3.80 (d, J=12.3 Hz, 3H), 4.87 (dd, J=36.4, 18.5 Hz, 2H), 6.97 (d, J=8.6 Hz, 1H), 7.14-7.34 (m, 6H), 7.38 (d, J=1.6 Hz, 1H); MS (APCI+) m/z 444 [M+1]⁺.

### Example 16

### 2-Amino-6-[2-(3-bromophenyl)ethyl]-3,6-dimethyl-5,6-dihydropyrimidin-4(3H)-one

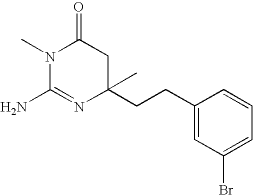

To a solution of 6-[2-(3-bromophenyl)ethyl]-2-imino-1-(4-methoxybenzyl)-3,6-dimethyltetrahydropyrimidin-4(1H)-one (20.27 g, 45.62 mmol) in acetonitrile (132 mL) was added water (33 mL) followed by ammonium cerium nitrate (75.03 g, 136.86 mmol) and the reaction was stirred for 18 h. Celite (40 g) was added followed by sodium bicarbonate (38.0 g, 452 mmol) and the reaction stirred for 1 h. Additional Celite (20 g) was added and the reaction filtered through Celite, the Celite was washed with acetonitrile and the filtrate was concentrated under reduced pressure. The resulting yellow gum was dissolved in ethanol, the salts removed by filtration, and solvent removed under reduced pressure. The resulting solids were triturated with diethyl ether and the tan solid put under high vacuum. The material was partitioned between ethyl acetate (500 mL) and a saturated sodium chloride/water solution (1:1, 500 mL). The organic layer was washed with aqueous saturated sodium chloride, dried over sodium sulfate, and the solvent was removed under reduced pressure. The crude material was chromatographed on a silica gel column eluting with dichloromethane/methanol/acetic acid (90:10:0.1), as the eluent. The solvents were removed under reduced pressure and the material placed under high vacuum over night to give 13.3 g (90% yield) of the title compound: ¹H NMR (300 MHz, DMSO-d₆/TFA-d) δ 1.33 (s, 3H), 1.86 (m, 2H), 2.64 (t, J=8.5 Hz, 2H), 2.79 (d, J=16.4 Hz, 1H), 2.94 (d, J=16.4 Hz, 1H), 3.20 (s, 3H), 7.26 (d, J=5.0 Hz, 2H), 7.40 (td, J=4.5, 1.9 Hz, 1H), 7.49 (s, 1H); MS (APCI+) m/z 324 [M+1]⁺.

### Example 17

### 2-Amino-6-[3-(5-chloro-2-fluoropyridin-3-yl)phenyl]-3,6-dimethyl-5,6-dihydropyrimidin-4(3H)-one hydrochloride

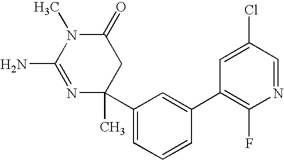

To a mixture of 2-amino-6-(3-bromophenyl)-3,6-dimethyl-5,6-dihydropyrimidin-4(3H)-one (100 mg, 0.338 mmol), tetrahydrofuran (2 mL) and water (200 μL) was added 5-chloro-2-fluoropyridine-3-boronic acid (83 mg, 0.473 mmol), sodium carbonate (125 mg, 1.18 mmol) and dichlorobis(triphenylphosphine)palladium(II) (30 mg, 0.0367 mmol). The reaction was irradiated an a microwave for 10 min at 140° C. When the mixture had reached room temperature, water (10 mL) was added and the mixture was extracted with ethyl acetate (20 mL). The phases were separated and the organic phase was washed with saturated aqueous sodium carbonate (20 mL), dried over calcium sulfate and concentrated under reduced pressure. The residue was purified using prep. HPLC using a XTerra column (19×300 mm) with 0.1% trifluoroacetic acid buffer/methanol (20-60% methanol), as the eluent. The purified fractions were combined and the acetonitrile was removed under reduced pressure. Saturated aqueous sodium hydrogen carbonate was added to the remaining solution until a precipitation was obtained and the mixture was extracted with ethyl acetate (20 mL). The organic phase was dried over calcium sulfate and then concentrated under reduced pressure to give a white solid. The solid was resolved in methanol (0.5 mL) and hydrochloric acid in diethyl ether (2 M, 200 μL) was added. Diethyl ether was added until a precipitation was obtained to give 35 mg (27% yield) of the title compound: ¹H NMR (400 MHz, DMSO-d₆) δ 10.78 (s, 1H) 8.85 (s, 2H) 8.27-8.49 (m, 2H) 7.48-7.80 (m, 4H) 3.50 (d, J=16.2 Hz, 1H) 3.21 (d, J=16.2 Hz, 1H) 3.08 (s, 3H) 1.64 (s, 3H); MS (AP+) m/z 347 [M+1]⁺.

The compounds in Examples 18-23 were synthesized as described for Example 17.

### Example 18

### 2-Amino-6-[3-(6-fluoro-5-methylpyridin-3-yl)phenyl]-3,6-dimethyl-5,6-dihydropyrimidin-4(3H)-one hydrochloride

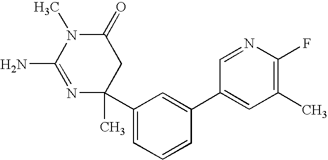

¹H NMR (400 MHz, DMSO-d₆) δ 10.71 (s, 1H) 8.82 (s, 2H) 8.39 (s, 1H) 8.23 (dd, J=9.6, 1.77 Hz, 1H) 7.83 (s, 1H) 7.67 (d, J=7.6 Hz, 1H) 7.52 (t, J=7.7 Hz, 1H) 7.44 (d, J=7.8 Hz, 1H) 3.55 (d, J=16.4 Hz, 1H) 3.21 (d, J=16.4 Hz, 1H) 2.31 (s, 3H) 3.07 (s, 3H) 1.64 (s, 3H); MS (ES+) m/z 327 [M+1]⁺.

### Example 19

### 2-Amino-6-[3-(2-chloropyridin-4-yl)phenyl]-3,6-dimethyl-5,6-dihydropyrimidin-4(3H)-one hydrochloride

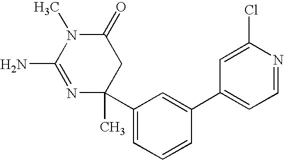

¹H NMR (400 MHz, DMSO-d₆) δ 10.84 (s, 1H) 8.88 (s, 2H) 8.49 (d, J=5.3 Hz, 1H) 7.96 (d, J=11.9 Hz, 2H) 7.78-7.86 (m, 2H) 7.53-7.60 (m, 2H) 3.58 (d, J=16.4 Hz, 1H) 3.22 (d, J=16.4 Hz, 1H) 3.07 (s, 3H) 1.64 (s, 3H); MS (ES+) m/z 329 [M+1]⁺.

### Example 20

### 2-Amino-6-{2-[3-(2-chloropyridin-4-yl)phenyl]ethyl}-3,6-dimethyl-5,6-dihydropyrimidin-4(3H)-one hydrochloride

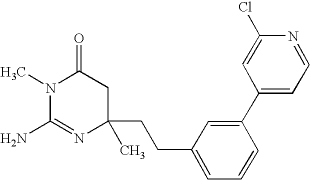

Starting from 2-amino-6-[2-(3-bromophenyl)ethyl]-3,6-dimethyl-5,6-dihydropyrimidin-4(3H)-one: ¹H NMR (400 MHz, DMSO-d₆) δ 9.92 (s, 1H) 8.46 (d, J=5.0 Hz, 3H) 7.85 (d, J=1.0 Hz, 1H) 7.72-7.79 (m, 2H) 7.68 (d, J=7.6 Hz, 1H) 7.44 (d, J=7.6 Hz, 1H) 7.38 (d, J=7.6 Hz, 1H) 2.89-2.98 (m, 1H) 3.16 (s, 3H) 2.78 (d, J=16.4 Hz, 1H) 2.68-2.76 (m, 2H) 1.90 (dd, J=11.2, 5.68 Hz, 2H) 1.32 (s, 3H); MS (ES+) m/z 357 [M+1]⁺.

### Example 21

### 2-Amino-6-[3-(5-methoxypyridin-3-yl)phenyl]-3,6-dimethyl-5,6-dihydropyrimidin-4(3H)-one hydrochloride

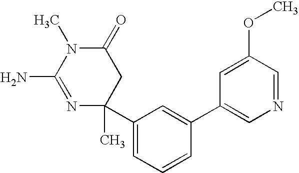

¹H NMR (400 MHz, DMSO-d₆) δ 10.91 (s, 1H) 8.91 (s, 2H) 8.70 (d, J=1.3 Hz, 1H) 8.46 (d, J=2.5 Hz, 1H) 8.03 (s, 1H) 7.96 (s, 1H) 7.79 (d, J=7.3 Hz, 1H) 7.49-7.60 (m, 2H) 3.99 (s, 3H) 3.56 (d, J=16.4 Hz, 1H) 3.24 (d, J=16.4 Hz, 1H) 3.08 (s, 3H) 1.65 (s, 3H); MS (ES+) m/z 325

### Example 22

### 2-Amino-6-{2-[3-(6-methoxypyridin-2-yl)phenyl]ethyl}-3,6-dimethyl-5,6-dihydropyrimidin-4(3H)-one hydrochloride

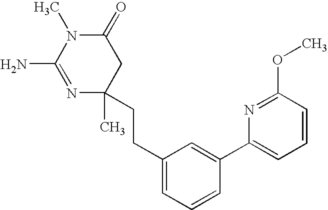

Starting from 2-amino-6-[2-(3-bromophenyl)ethyl]-3,6-dimethyl-5,6-dihydropyrimidin-4(3H)-one: ¹H NMR (400 MHz, DMSO-d₆) δ 10.11 (s, 1H) 8.54 (s, 2H) 7.88-7.98 (m, 2H) 7.72-7.81 (m, 1H) 7.54 (d, J=7.3 Hz, 1H) 7.40 (t, J=7.6 Hz, 1H) 7.30 (d, J=7.6 Hz, 1H) 6.77 (d, J=8.3 Hz, 1H) 3.95 (s, 3H) 3.16 (s, 3H) 2.91-2.99 (m, 1H) 2.70-2.82 (m, 3H) 1.82-1.94 (m, 2H) 1.32 (s, 3H); MS (ES+) m/z 353 [M+1]⁺.

### Example 23

### 2-Amino-6-{2-[3-(5-chloro-2-fluoropyridin-3-yl)phenyl]ethyl}-3,6-dimethyl-5,6-dihydropyrimidin-4(3H)-one hydrochloride

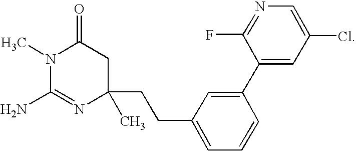

Starting from 2-amino-6-[2-(3-bromophenyl)ethyl]-3,6-dimethyl-5,6-dihydropyrimidin-4(3H)-one: ¹H NMR (400 MHz, DMSO-d₆) δ 10.11 (s, 1H) 8.54 (s, 2H) 8.31 (d, J=1.3 Hz, 1H) 8.27 (dd, J=8.6, 2.5 Hz, 1H) 7.53 (s, 1H) 7.41-7.49 (m, 2H) 7.33-7.38 (m, 1H) 3.16 (s, 3H) 2.88-2.96 (m, 1H) 2.69-2.81 (m, 3H) 1.84-1.93 (m, 2H) 1.31 (s, 3H); MS (ES+) m/z (375) [M+1]⁺.

### Example 24

### (6-Methoxypyridin-2-yl)boronic acid

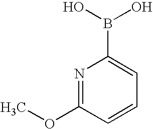

2-Bromo-6-methoxypyridine (2 g, 10.6 mmol) and triisopropylborate (2.4 g, 12.8 mmol) were mixed in toluene (15 mL). The mixture was cooled to −78° C. and n-butyllithium in hexane (2.5 M, 5.1 mL, 12.76 mmol) was added dropwise during 10 min. The mixture was stirred for 5 h at −78° C., allowed to warm to −20° C. and aqueous hydrochloric acid (2 M, 10 mL) was added. The organic and aqueous layers were separated and the aqueous layer was acidified to pH 4 with aqueous hydrobromide (48%). The title compound 0.13 g (8% yield) was filtered off and used as is directly in the next step without further characterization.

### Example 25

### (5-Methoxypyridin-3-yl)boronic acid

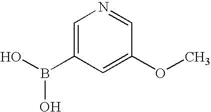

The title compound was synthesized as described for Example 24 starting from 3-bromo-5-methoxypyridine and was used in the next step without further characterization.

### Assays

Compounds were tested in at least one of the following assays:

### β-Secretase Enzyme

The enzyme used in the IGEN Cleavage-, Fluorescent-, TR-FRET- and the BiaCore assay is described as follows:

The soluble part of the human β-Secretase (AA 1-AA 460) was cloned into the ASP2-Fc10-1-IRES-GFP-neoK mammalian expression vector. The gene was fused to the Fc domain of IgG1 (affinity tag) and stably cloned into HEK 293 cells. Purified sBACE-Fc is stored in Tris buffer, pH 9.2 and has a purity of 95%.

### IGEN Cleavage Assay

Enzyme is diluted 1:30 in 40 mM MES pH 5.0. Stock substrate is diluted to 12 μM in 40 mM MES pH 5.0. Compounds are diluted to the desired concentration in dimethylsulphoxide (final dimethylsulphoxide concentration in assay is 5%). The assay is done in a 96 well PCR plate from Greiner (#650201). Compound in dimethylsulphoxide (3 μL) is added to the plate, and then enzyme is added (27 μL) and pre-incubated with compound for 10 minutes. The reaction is started with substrate (30 μL). The final dilution of enzyme is 1:60 and the final concentration of substrate is 6 μM. After a 20 minute reaction at room temperature, the reaction is stopped by removing 10 μl of the reaction mix and diluting it 1:25 in 0.2 M Trizma-HCl, pH 8.0. Compounds are diluted and added to the plate by the Biomek FX or by hand, then all the rest of the liquid handling is done with on the Biomek 2000 instrument.

All antibodies and the streptavidin coated beads are diluted in PBS containing 0.5% BSA and 0.5% Tween20. The product is quantified by adding 50 μL of a 1:5000 dilution of the neoepitope antibody to 50 μL of the 1:25 dilution of the reaction mix. Then, 100 μL of PBS (0.5% BSA, 0.5% Tween20) containing 0.2 mg/mL IGEN beads (Dynabeads M-280) and a 1:5000 dilution of ruthinylated goat anti-rabbit (Ru-GαR) antibody is added. The final dilution of neoepitope antibody is 1:20,000, the final dilution of Ru-GAR is 1:10,000 and the final concentration of beads is 0.1 mg/mL. The mixture is read on the IGEN instrument (BioVeris) with the Abbiochemial assay program after a 2-hour incubation with shaking at room temperature.

### Fluorescent Assay

Enzyme is diluted 1:25 in 40 mM MES pH 5.0. Stock substrate (Dabcyl) is diluted to 30 μM in 40 mM MES pH 5.0. Enzyme and substrate stock solutions are kept on ice until placed in the stock plates. The Biomek FX instrument is used to do all liquid handling. Enzyme (9 μL) together with 1 μL of compound in dimethylsulphoxide is added to the plate and pre-incubated for 10 minutes. When a dose response curve is being tested for a compound, the dilutions are done in neat dimethylsulphoxide. Substrate (10 μL) is added and the reaction proceeds in the dark for 25 minutes at room temperature. The assay is done in a Corning 384 well round bottom, low volume, non-binding surface (Corning #3676). The final dilution of enzyme is 1:50, and the final concentration of substrate is 15 μM (Km of 25 μM). The fluorescence of the product is measured on a Victor II plate reader with an excitation wavelength of 360 nm and an emission wavelength of 485 nm using the protocol for labelled Edans peptide. The dimethylsulphoxide control defines 100% activity level and 0% activity is defined by exclusion of the enzyme (using 40 mM MES pH 5.0 buffer instead).

### TR-FRET Assay

Dilute the enzyme (truncated form) to 6 μg/mL (stock 1.3 mg/mL) and the substrate (Europium)CEVNLDAEFK(Qsy7) to 200 nM (stock 60 μM) in reaction buffer (NaAcetate, chaps, triton x-100, EDTA pH4.5). The Biomek FX is used for all liquid handling and the enzyme and substrate solutions are kept on ice until they are placed in Biomek FX. Enzyme (9 μl) is added to the plate then 1 μl of compound in dimethylsulphoxide is added, mixed and pre-incubated for 10 minutes. Substrate (10 μl) is then added, mixed and the reaction proceeds in the dark for 15 minutes at room temperature. The reaction is stopped with the addition of Stop solution (7 μl, NaAcetate pH 9). The fluorescence of the product is measured on a Victor II plate reader with an excitation wavelength of 340 nm and an emission wavelength of 615 nm. The assay is done in a Costar 384 well round bottom, low volume, non-binding surface (Corning #3676). The final concentration of the enzyme is 0.3 nM; the final concentration of substrate is 100 nM (Km of 250 nM). The dimethylsulphoxide control defines the 100% activity level and 0% activity is defined by only addition of the peptide substrate. A control inhibitor is also used in dose response assays and has an IC₅₀ of 575 nM.

### Beta-Secretase Whole Cell Assay

### Generation of HEK293-APP695

The pcDNA3.1 plasmid encoding the cDNA of human full-length APP695 was stably transfected into HEK-293 cells using the Lipofectamine transfection reagent according to manufacture's protocol (Invitrogen). Colonies were selected with 0.1-0.5 mg/mL of zeocin. Limited dilution cloning was performed to generate homogeneous cell lines. Clones were characterized by levels of APP expression and Aβ secreted in the conditioned media using an ELISA assay developed in-house.

### Cell Culture

HEK293 cells stably expressing human wild-type APP (HEK293-APP695) were grown at 37° C. in DMEM containing 4500 g/L glucose, GlutaMAX and sodium pyruvate supplemented with 10% FBS, 1% non-essential amino acids and 0.1 mg/mL of the selection antibiotic zeocin.

### Aβ40 Release Assay

Cells were harvested at 80-90% confluence and seeded at a concentration of 0.2×10⁶ cells/mL, 100 mL cell suspension/well, onto a black clear bottom 96-well poly-D-lysine coated plate. After over night incubation at 37° C., 5% CO₂, the cell medium was replaced with cell culture medium with penicillin and streptomycin (100 U/mL, 100 μg/mL, respectively) and containing test compounds in a final dimethylsulphoxide concentration of 1%. Cells were exposure to test compounds for 24 h at 37° C., 5% CO2. To quantify the amount of released Aβ, 100 μL cell medium was transferred to a round bottom polypropylene 96-well plate (assay plate). The cell plate was saved for ATP assay as described in ATP assay below. To the assay plate, 50 μL of primary detection solution containing 0.5 μg/mL of the rabbit anti-Aβ40 antibody and 0.5 μg/mL of the biotinylated monoclonal mouse 6E10 antibody in DPBS with 0.5% BSA and 0.5% Tween-20 was added per well and incubated over night at 4° C. Then, 50 μL of secondary detection solution containing 0.5 μg/mL of a ruthenylated goat anti-rabbit antibody and 0.2 mg/mL of streptavidin coated Dynabeads was added per well. The plate was vigorously shaken at room temperature for 1-2 h. The plate was then measured for electro-chemiluminescence counts in an IGEN M8 Analyzer. An Aβ standard curve was obtained using standards at concentrations 20, 10, 2 and 0.2 ng Aβ/mL in the cell culture medium with penicillin and streptomycin (100 U/mL, 100 μg/mL, respectively).

### ATP Assay

As indicated above, after transferring 100 μL medium from the cell plate for Aβ40 detection, the plate was used to analyse cytotoxicity using the ViaLight™ Plus cell proliferation/cytotoxicity kit from Cambrex BioScience that measures total cellular ATP. The assay was performed according to the manufacture's protocol. Briefly, 50 μL cell lysis reagent was added per well. The plates were incubated at room temperature for 10 min. Two min after addition of 100 μL reconstituted ViaLight™ Plus ATP reagent, the luminescence was measured in a Wallac Victor² 1420 multilabel counter.

### BACE Biacore Protocol

### Sensor Chip Preparation

BACE was assayed on a Biacore3000 instrument by attaching either a peptidic transition state isostere (TSI) or a scrambled version of the peptidic TSI to the surface of a Biacore CM5 sensor chip. The surface of a CM5 sensor chip has 4 distinct channels that can be used to couple the peptides. The scrambled peptide KFES-statine-ETIAEVENV was coupled to channel 1 and the TSI inhibitor KTEEISEVN-statine-VAEF was couple to channel 2 of the same chip. The two peptides were dissolved at 0.2 mg/mL in 20 mM Na-Acetate pH 4.5, and then the solutions were centrifuged at 14K rpm to remove any particulates. Carboxyl groups on the dextran layer were activated by injecting a one to one mixture of 0.5 M N-ethyl-N′ (3-dimethylaminopropyl)-carbodiimide (EDC) and 0.5 M N-hydroxysuccinimide (NHS) at 5 uL/minute for 7 minutes. Then the stock solution of the control peptide was injected in channel 1 for 7 minutes at 5 uL/min., and then the remaining activated carboxyl groups were blocked by injecting 1M ethanolamine for 7 minutes at 5 uL/minute.

### Assay Protocol

The BACE Biacore assay was done by diluting BACE to 0.5 μM in Na Acetate buffer at pH 4.5 (running buffer minus dimethylsulphoxide). The diluted BACE was mixed with dimethylsulphoxide or compound diluted in dimethylsulphoxide at a final concentration of 5% dimethylsulphoxide. The BACE/inhibitor mixture was incubated for 1 hour at 4° C. then injected over channel 1 and 2 of the CM5 Biacore chip at a rate of 20 μL/minute. As BACE bound to the chip the signal was measured in response units (RU). BACE binding to the TSI inhibitor on channel 2 gave a certain signal. The presence of a BACE inhibitor reduced the signal by binding to BACE and inhibiting the interaction with the peptidic TSI on the chip. Any binding to channel 1 was non-specific and was subtracted from the channel 2 responses. The dimethylsulphoxide control was defined as 100% and the effect of the compound was reported as percent inhibition of the dimethylsulphoxide control.

hERG Assay

### Cell Culture

The hERG-expressing Chinese hamster ovary K1 (CHO) cells described by (Persson, Carlsson, Duker, & Jacobson, 20.05) were grown to semi-confluence at 37° C. in a humidified environment (5% CO₂) in F-12 Ham medium containing L-glutamine, 10% foetal calf serum (FCS) and 0.6 mg/ml hygromycin (all Sigma-Aldrich). Prior to use, the monolayer was washed using a pre-warmed (37° C.) 3 ml aliquot of Versene 1:5,000 (Invitrogen). After aspiration of this solution the flask was incubated at 37° C. in an incubator with a further 2 ml of Versene 1:5,000 for a period of 6 minutes. Cells were then detached from the bottom of the flask by gentle tapping and 10 ml of Dulbecco's Phosphate-Buffered Saline containing calcium (0.9 mM) and magnesium (0.5 mM) (PBS; Invitrogen) was then added to the flask and aspirated into a 15 ml centrifuge tube prior to centrifugation (50 g, for 4 mins). The resulting supernatant was discarded and the pellet gently re-suspended in 3 ml of PBS. A 0.5 ml aliquot of cell suspension was removed and the number of viable cells (based on trypan blue exclusion) was determined in an automated reader (Cedex; Innovatis) so that the cell re-suspension volume could be adjusted with PBS to give the desired final cell concentration. It is the cell concentration at this point in the assay that is quoted when referring to this parameter. CHO-Kv1.5 cells, which were used to adjust the voltage offset on IonWorks™ HT, were maintained and prepared for use in the same way.

### Electrophysiology

The principles and operation of this device have been described by (Schroeder, Neagle, Trezise, & Worley, 2003). Briefly, the technology is based on a 384-well plate (PatchPlate™) in which a recording is attempted in each well by using suction to position and hold a cell on a small hole separating two isolated fluid chambers. Once sealing has taken place, the solution on the underside of the PatchPlate™ is changed to one containing amphotericin B. This permeablises the patch of cell membrane covering the hole in each well and, in effect, allows a perforated, whole-cell patch clamp recording to be made.

A β-test IonWorks™ HT from Essen Instrument was used. There is no capability to warm solutions in this device hence it was operated at room temperature (˜21° C.), as follows. The reservoir in the “Buffer” position was loaded with 4 ml of PBS and that in the “Cells” position with the CHO-hERG cell suspension described above. A 96-well plate (V-bottom, Greiner Bio-one) containing the compounds to be tested (at 3-fold above their final test concentration) was placed in the “Plate 1” position and a PatchPlate™ was clamped into the PatchPlate™ station. Each compound plate was laid-out in 12 columns to enable ten, 8-point concentration-effect curves to be constructed; the remaining two columns on the plate were taken up with vehicle (final concentration 0.33% DMSO), to define the assay baseline, and a supra-maximal blocking concentration of cisapride (final concentration 10 μM) to define the 100% inhibition level. The fluidics-head (F-Head) of IonWorks™ HT then added 3.5 μl of PBS to each well of the PatchPlate™ and its underside was perfused with “internal” solution that had the following composition (in mM): K-Gluconate 100, KCl 40, MgCl₂ 3.2, EGTA 3 and HEPES 5 (all Sigma-Aldrich; pH 7.25-7.30 using 10 M KOH). After priming and de-bubbling, the electronics-head (E-head) then moved round the PatchPlate™ performing a hole test (i.e. applying a voltage pulse to determine whether the hole in each well was open). The F-head then dispensed 3.5 μl of the cell suspension described above into each well of the PatchPlate™ and the cells were given 200 seconds to reach and seal to the hole in each well. Following this, the E-head moved round the PatchPlate™ to determine the seal resistance obtained in each well. Next, the solution on the underside of the PatchPlate™ was changed to “access” solution that had the following composition (in mM): KCl 140, EGTA 1, MgCl₂ 1 and HEPES 20 (pH 7.25-7.30 using 10 M KOH) plus 100 μg/ml of amphotericin B (Sigma-Aldrich). After allowing 9 minutes for patch perforation to take place, the E-head moved round the PatchPlate™ 48 wells at a time to obtain pre-compound hERG current measurements. The F-head then added 3.5 μl of solution from each well of the compound plate to 4 wells on the PatchPlate™ (the final DMSO concentration was 0.33% in every well). This was achieved by moving from the most dilute to the most concentrated well of the compound plate to minimise the impact of any compound carry-over. After approximately 3.5 mins incubation, the E-head then moved around all 384-wells of the PatchPlate™ to obtain post-compound hERG current measurements. In this way, non-cumulative concentration-effect curves could be produced where, providing the acceptance criteria were achieved in a sufficient percentage of wells (see below), the effect of each concentration of test compound was based on recording from between 1 and 4 cells.

The pre- and post-compound hERG current was evoked by a single voltage pulse consisting of a 20 period holding at −70 mV, a 160 ms step to −60 mV (to obtain an estimate of leak), a 100 ms step back to −70 mV, a 1 s step to +40 mV, a 2 s step to −30 mV and finally a 500 ms step to −70 mV. In between the pre- and post-compound voltage pulses there was no clamping of the membrane potential. Currents were leak-subtracted based on the estimate of current evoked during the +10 mV step at the start of the voltage pulse protocol. Any voltage offsets in IonWorks™ HT were adjusted in one of two ways. When determining compound potency, a depolarising voltage ramp was applied to CHO-Kv1.5 cells and the voltage noted at which there was an inflection point in the current trace (i.e. the point at which channel activation was seen with a ramp protocol). The voltage at which this occurred had previously been determined using the same voltage command in conventional electrophysiology and found to be −15 mV (data not shown); thus an offset potential could be entered into the IonWorks™ HT software using this value as a reference point. When determining the basic electrophysiological properties of hERG, any offset was adjusted by determining the hERG tail current reversal potential in IonWorks™ HT, comparing it with that found in conventional electrophysiology (−82 mV) and then making the necessary offset adjustment in the IonWorks™ HT software. The current signal was sampled at 2.5 kHz.

Pre- and post-scan hERG current magnitude was measured automatically from the leak subtracted traces by the IonWorks™ HT software by taking a 40 ms average of the current during the initial holding period at −70 mV (baseline current) and subtracting this from the peak of the tail current response. The acceptance criteria for the currents evoked in each well were: pre-scan seal resistance >60 MΩ, pre-scan hERG tail current amplitude >150 pA; post-scan seal resistance >60 MΩ. The degree of inhibition of the hERG current was assessed by dividing the post-scan hERG current by the respective pre-scan hERG current for each well.

### Results

Typical IC₅₀ values for the compounds of the present invention are in the range of about 1 to about 10,000 nM. Biological data on examples is given below in Table 1.

TABLE 1

| Example No. | IC50 in TR-FRET Assay |
|---|---|
| Example 20 | 114 nM |
| Example 21 | 316 nM |

### SEARCH HISTORY:

### IPC Classification: C07D401/10, A61K31/513, A61P25/28

### ECLA Classification: C07D401/10, A61K31/513

### Internal:

Search terms used: amyloid, angiopathy, downs, hemorrhage, cognitive, alzheimer, memory, deficit, neurodegeneration, dementia, senile, parkinson, palsy, cortical

### STN:

| | FILE ‘REGISTRY’ ENTERED AT 10:15:45 ON 13 OCT 2006 | | |
|---|---|---|---|
| L1 | | | STRUCTURE UPLOADED |
| L2 | | | SCREEN 1840 |
| L3 | | | STRUCTURE UPLOADED |
| L4 | | | QUE L3 AND L2 |
| L5 | | 59692 | S L1 FULL |
| | | | SAVE L5 TEMP PCT153240/A |
| | FILE ‘CAPLUS’ ENTERED AT 10:31:02 ON 13 OCT 2006 | | |
| | | | SET EXP CONT |
| | | | SET PLUR ON |
| | | | SET ABB ON |
| | | | E DOWNS |
| | | | E AMYLOID |
| | | | E ANGIOPATHY |
| L6 | | 218 | S L5 AND (E3 OR E15 OR E27) |
| L7 | | 6 | S L5 AND E3 |
| L8 | | 12 | S L5 AND (E15 AND E27) |
| L9 | | 12 | S L8 NOT L7 |
| | FILE ‘REGISTRY’ ENTERED AT 10:35:35 ON 13 OCT 2006 | | |
| L10 | | 18 | S L4 SUB=L5 SAM |
| L11 | | 437 | S L4 FULL SUB=L5 |
| | FILE ‘ZREGISTRY’ ENTERED AT 10:37:29 ON 13 OCT 2006 | | |
| L12 | | | SCREEN 1840 |
| L13 | | | STRUCTURE UPLOADED |
| L14 | | | QUE L13 AND L12 |
| | FILE ‘REGISTRY’ ENTERED AT 10:38:58 ON 13 OCT 2006 | | |
| L15 | | 15 | S L14 SAM SUB=L5 |
| L16 | | 218 | S L14 FULL SUB=L5 |
| | FILE ‘CAPLUS’ ENTERED AT 10:40:52 ON 13 OCT 2006 | | |
| L17 | | 4 | S L16 |
| L18 | | 2 | S L17 NOT (L9 OR L7) |

### Structure L1:

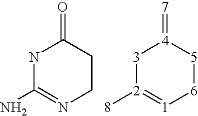

chain nodes:
7 8
ring nodes:
1 2 3 4 5 6
chain bonds:
2-8 4-7
ring bonds:
1-2 1-6 2-3 3-4 4-5 5-6
exact/norm bonds:
1-2 1-6 2-3 2-8 3-4 4-5 4-7 5-6
Match level:

### 1:Atom 2:Atom 3:Atom 4:Atom 5:Atom 6:Atom 7:CLASS 8:CLASS

### Structure: L3:

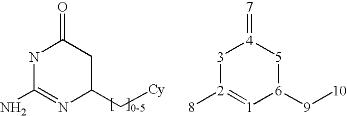

chain nodes
7 8 9 10
ring nodes
1 2 3 4 5 6
chain bonds:
2-8 4-7 6-9 9-10
ring bonds:
1-2 1-6 2-3 3-4 4-5 5-6
exact/norm bonds:
1-2 1-6 2-3 2-8 3-4 4-5 4-7 5-6 9-10
exact bonds:
6-9
Match level:

### 1:Atom 2:Atom 3:Atom 4:Atom 5:Atom 6:Atom 7:CLASS 8:CLASS 9:CLASS 10:Atom

Generic attributes:
10:

### Saturation: Unsaturated

### Structure L13:

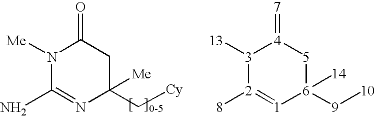

chain nodes:
7 8 9 10 13 14
ring nodes:
1 2 3 4 5 6
chain bonds:
2-8 3-13 4-7 6-9 6-14 9-10
ring bonds:
1-2 1-6 2-3 3-4 4-5 5-6
exact/norm bonds:
1-2 1-6 2-3 2-8 3-4 4-5 4-7 5-6 9-10
exact bonds:
3-13 6-9 6-14
Match level:

### 1:Atom 2:Atom 3:Atom 4:Atom 5:Atom 6:Atom 7:CLASS 8:CLASS 9:CLASS 10:Atom 13:CLASS 14:CLASS

Generic attributes:
10:

### Saturation: Unsaturated

### RESULTS:

| Derwent/ | Patent No/ | Patentee/ | | | | | |
|---|---|---|---|---|---|---|---|
| CAS Acc No | Source | Author | Title | Comment | X | Y | A |
| 144:412533 | WO 2006041404 | Astrazeneca | Preparation of substituted 2- | Similar compounds as | PX | | |
| | A1 | AB, Swed.; | aminopyrimidin-4-ones for | well as compounds | 1-28 | | |
| | | Astex | treating or preventing Aβ- | that falls within | | | |
| | | Therapeutics | related pathologies. | the scope of the | | | |
| | | | | claims (RN: 883891- | | | |
| | | | | 38-1, 883891-47-2) | | | |
| | | | | that have the same | | | |
| | | | | pharmaceutical use. | | | |
| 144:412528 | WO 2006041405 | Astrazeneca | Preparation of substituted 2- | Similar compounds as | PX | | |
| | A1 | AB, Swed.; | aminopyrimidin-4-ones for | well as compounds | 1-28 | | |
| | | Astex | treating or preventing Aβ- | that falls within | | | |
| | | Therapeutics | related pathologies. | the scope of the | | | |
| | | | | claims (RN: 883891- | | | |
| | | | | 38-1, 883891-47-2) | | | |
| | | | | that have the same | | | |
| | | | | pharmaceutical use. | | | |
| 145:8167 | WO 2006065277 | Schering | Preparation of imidazolidin-2- | Similar compounds as | PX | | |
| | A2 | Corporation, | imines and their analogs as | well as compounds | 1-28 | | |
| | | USA; | aspartyl protease inhibitors | that falls within | | | |
| | | Pharmacopeia | for treating various diseases. | the scope of the | | | |
| | | Drug | | claims (RN: 887911- | | | |
| | | Discovery, | | 22-0) that have the | | | |
| | | Inc. | | same pharmaceutical | | | |
| | | | | use. | | | |
| 143:97365 | WO 2005058311 | Schering | Preparation of heterocyclic | Similar compounds | 1-28 | | |
| | A1 | Corporation, | aspartyl protease inhibitors | (RN: 856875-82-6, | | | |
| | | USA; | for treating various diseases. | 856875-83-7) that | | | |
| | | Pharmacopeia | | are used in the | | | |
| | | Drug | | treatment of | | | |
| | | Discovery, | | cognitive, | | | |
| | | Inc. | | neurodegenerative | | | |
| | | | | diseases and | | | |
| | | | | Alzheimers. | | | |
| 132:265206 | WO 2000018758 | Mitsubishi | Preparation of pyrimidones for | Related compounds | | | 1-28 |
| | A1 | Chemical | treating diseases caused by tau | used in the | | | |
| | | Corporation, | protein kinase 1 hyperactivity | treatment of | | | |
| | | Japan | such as Alzheimer disease. | Alzheimer disease, | | | |
| | | | | Down syndrome, | | | |
| | | | | cerebral bleeding | | | |
| | | | | due to cerebral | | | |
| | | | | amyloid angiopathy | | | |
| | | | | and progressive | | | |
| | | | | supranuclear palsy. | | | |

Examiners remarks:

1. A compound of formula I: 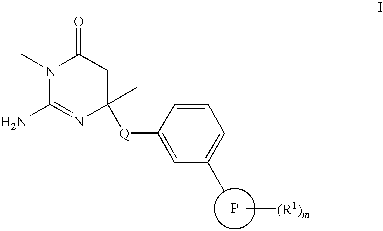
wherein
P is a pyridine ring;
Q is a bond or CH₂CH₂;
R¹ is independently selected from cyano, halogen, C₁₋₆alkyl and methoxy;
m is 1 or 2;
with the proviso that the following compounds are excluded;
2-Amino-6-[3-(5-bromopyridin-3-yl)phenyl]-3,6-dimethyl-5,6-dihydropyrimidin-4(3H)-one;
2-Amino-6-[3-(2-fluoropyridin-3-yl)phenyl]-3,6-dimethyl-5,6-dihydropyrimidin-4(3H)-one;
2-Amino-6-[3-(2-chloro-3-fluoropyridin-4-yl)phenyl]-3,6-dimethyl-5,6-dihydropyrimidin-4(3H)-one;
as a free base or a pharmaceutically acceptable salt, solvate or solvate of a salt thereof.

2. A compound according to claim 1, wherein C₁₋₆alkyl represents methyl.

3. A compound according to claim 1, wherein Q represents a direct bond.

4. A compound according to claim 1, wherein m is 1 and R¹ is cyano, methoxy or halogen.

5. A compound according to claim 4, wherein R¹ is halogen, said halogen being chloro attached in the 2-position in the pyridine ring.

6. A compound according to claim 2, wherein m is 2 and R¹ represents one halogen and one methyl.

7. A compound according to claim 6, wherein said halogen is fluoro attached in the 6-position in the pyridine ring.

8. A compound according to claim 2, wherein m is 2 and R¹ represents two halogen atoms.

9. A compound according to claim 8, wherein said two halogen atoms represents either of the following combinations of halogens: chloro attached in the 2- and 3-position in the pyridine ring; fluoro attached in the 2-position and bromo attached in the 5-position in the pyridine ring; chloro attached in the 2-position and fluoro attached in the 5-position in the pyridine ring; fluoro attached in the 2-position and chloro attached in the 5-position in the pyridine ring.

10. A compound according to claim 2, wherein Q represents CH₂—CH₂.

11. A compound according to claim 10, wherein m is 1 and R¹ is selected from halogen or methoxy.

12. A compound according to claim 11, wherein said halogen is chloro attached in the 2-position in the pyridine ring.

13. A compound according to claim 10, wherein m is 2 and R¹ represents two halogen atoms.

14. A compound according to claim 13, wherein said two halogen atoms represents fluoro attached in the 2-position and chloro attached in the 5-position in the pyridine ring.

15. A compound selected from:
5-[3-(2-Amino-1,4-dimethyl-6-oxo-1,4,5,6-tetrahydropyrimidin-4-yl)phenyl]nicotinonitrile hydrochloride;
2-Amino-6-[3-(2,3-dichloropyridin-4-yl)phenyl]-3,6-dimethyl-5,6-dihydropyrimidin-4(3H)-one acetate;
2-Amino-6-[3-(5-bromo-2-fluoropyridin-3-yl)phenyl]-3,6-dimethyl-5,6-dihydropyrimidin-4(3H)-one acetate;
2-Amino-6-[3-(2-chloro-5-fluoropyridin-4-yl)phenyl]-3,6-dimethyl-5,6-dihydropyrimidin-4(3H)-one trifluoroacetic acid salt;
2-Amino-6-[3-(5-chloro-2-fluoropyridin-3-yl)phenyl]-3,6-dimethyl-5,6-dihydropyrimidin-4(3H)-one hydrochloride;
2-Amino-6-[3-(6-fluoro-5-methylpyridin-3-yl)phenyl]-3,6-dimethyl-5,6-dihydropyrimidin-4(3H)-one hydrochloride;
2-Amino-6-[3-(2-chloropyridin-4-yl)phenyl]-3,6-dimethyl-5,6-dihydropyrimidin-4(3H)-one hydrochloride;
2-Amino-6-{2-[3-(2-chloropyridin-4-yl)phenyl]ethyl}-3,6-dimethyl-5,6-dihydropyrimidin-4(3H)-one hydrochloride;
2-Amino-6-[3-(5-methoxypyridin-3-yl)phenyl]-3,6-dimethyl-5,6-dihydropyrimidin-4(3H)-one hydrochloride;
2-Amino-6-{2-[3-(6-methoxypyridin-2-yl)phenyl]ethyl}-3,6-dimethyl-5,6-dihydropyrimidin-4(3H)-one hydrochloride; and
2-Amino-6-{2-[3-(5-chloro-2-fluoropyridin-3-yl)phenyl]ethyl}-3,6-dimethyl-5,6-dihydropyrimidin-4(3H)-one hydrochloride;
as a free base or a pharmaceutically acceptable salt, solvate or solvate of a salt thereof.

16. A pharmaceutical composition comprising as active ingredient a therapeutically effective amount of a compound according to claim 1 in association with a pharmaceutically acceptable excipient, carrier or diluent.

17-21. (canceled)

22. A method of inhibiting activity of BACE comprising contacting said BACE with a compound of claim 1.

23. A method of treating or preventing an Aβ-related pathology in a mammal, comprising administering to said patient a therapeutically effective amount of a compound of claim 1.

24. The method of claim 23, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

25. The method of claim 23, wherein said mammal is a human.

26. A method of treating or preventing an Aβ-related pathology in a mammal, comprising administering to said patient a therapeutically effective amount of a compound of claim 1 and at least one cognitive enhancing agent, memory enhancing agent, or choline esterase inhibitor.

27. The method of claim 26, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

28. The method of claim 26, wherein said mammal is a human.


SEQUENCE LISTING (3 sequences)

SEQ ID NO: 1 - protein, 10 aa, Artificial Sequence
  TR-FRET Assay substrate.
CEVNLDAEFK

SEQ ID NO: 2 - protein, 14 aa, Artificial Sequence
  BACE assay scrambled peptide.
KFESXETIAEVENV

SEQ ID NO: 3 - protein, 14 aa, Artificial Sequence
  TSI assay peptide
KTEEISEVNXVAEF